(19) United States
(12) Patent Application Publication
(10) Pub. No.: US 2017/0292010 A1
(43) Pub. Date: Oct. 12, 2017

(54) AQUEOUS ETHYLCELLULOSE DISPERSIONS With polymeric additive

(71) Applicant: Dow Global Technologies LLC, Midland, MI (US)

(72) Inventors: David L. Malotky, Midland, MI (US); Shari L. Workentine, Midland, MI (US); True L. Rogers, Midland, MI (US); Stacey L. Dean, Litchfield, OH (US); Matthew J. Crimmins, Saint Charles, MI (US)

(21) Appl. No.: 15/512,860

(22) PCT Filed: Sep. 28, 2015
(86) PCT No.: PCT/US15/52581

§ 371 Date: Mar. 21, 2017

Related U.S. Application Data
(60) Provisional application No. 62/057,279, filed on Sep. 30, 2014.

(51) Int. Cl. C08L 1/28; A61K 9/20; C09D 7/02; C08J 5/18; C09D 101/28; C09D 7/12; A61K 9/28; A61K 9/48

(52) U.S. Cl. CPC C08L 1/28; A61K 9/28; A61K 9/2054; A61K 9/4891; A61K 9/4866; C08J 5/18; C09D 101/28; C09D 7/125; C09D 7/02; C08L 2203/16; C08L 2203/02; C08J 2301/28; C08L 2201/50

(57) ABSTRACT

Provided is an aqueous composition having pH of 8 or higher and comprising (a) a solid phase comprising dispersed particles that comprise an amount of ethylcellulose polymer, (b) an amount of one or more polymeric dispersants, wherein said polymeric dispersant has a weight-average molecular weight of 5,000 daltons or higher, and wherein said polymeric dispersant has an acid value of 60 to 190 mg KOH/g of polymer. Also provided is a method of making such a composition using an extruder. Also provided is a film made by removing water from such a composition.

It is often desirable to make films that contain ethylcellulose polymer. Such films are useful, for example, as coatings applied to other films or to beads. In some cases, a collection of beads contains a drug, and each of those beads is then coated with a film that contains ethylcellulose polymer. The film that contains ethylcellulose polymer can provide controlled release of the drug when the beads are placed in an aqueous environment such as that which can be found in the human body. It is also desirable that the film have good mechanical properties such as high tensile strength, high tensile elongation, and/or surface smoothness. In the past, a common method of making such a film was to contact the beads with a solution in which ethylcellulose polymer was dissolved in an organic solvent. Organic solvents are undesirable because of environmental and health effects. It is desired to provide an aqueous coating composition that contains ethylcellulose polymer and that is capable of producing high quality films. One desirable form of such an aqueous coating composition is an aqueous ethylcellulose polymer dispersion, which is a form in which particles of ethylcellulose polymer are dispersed in a continuous aqueous medium.

In the past, some dispersions were made using relatively high levels of monomeric dispersants. Such monomeric dispersants can lower some or all of the mechanical properties of films made from such dispersions, and such monomeric dispersants can lower the storage stability of such dispersions. In some cases, monomeric dispersants can negatively affect health, safety, or environmental issues. U.S. Pat. No. 4,502,888 describes dispersions of water-insoluble polymers that employ fatty acid salts as plasticizers/stabilizers. It is desired to provide aqueous dispersions of ethylcellulose that employ polymeric dispersants.

The following is a statement of the invention.

A first aspect of the present invention is an aqueous composition having pH of 8 or higher and comprising
(a) a solid phase comprising dispersed particles that comprise an amount of ethylcellulose polymer,
(b) an amount of one or more polymeric dispersants, wherein said polymeric dispersant has weight-average molecular weight of 5,000 daltons or higher, and wherein said polymeric dispersant has an acid value of 60 to 190 mg KOH/g of polymer.

A second aspect of the present invention is a method of making the aqueous composition of the first aspect, wherein said method comprises
(i) feeding the ethylcellulose polymer and the polymeric dispersant into a melt and mix zone of an extruder wherein the ethylcellulose polymer and the polymeric dispersant are heated and mixed together to form a melt;
(ii) conveying the melt to an emulsification zone of the extruder in which the temperature and pressure are controlled;
(iii) feeding a base and water into the emulsification zone wherein the melt is dispersed to form a concentrated emulsion having 60% to 72% solids by volume;
(iv) conveying the emulsion to a dilution and cooling zone of the extruder; and feeding water into the dilution and cooling zone to dilute the concentrated emulsion thereby forming the aqueous composition.

A third aspect of the present invention is a film made by a process comprising removing water from the aqueous composition of the first aspect.

The following is a detailed description of the invention.

As used herein, the following terms have the designated definitions, unless the context clearly indicates otherwise.

As used herein, an aqueous composition has 20% or more water by weight based on the weight of the composition. As used herein, a dispersion is a composition that contains a continuous medium that is liquid at 25° C. and contains discrete particles (herein called the “dispersed particles”) of a substance that are distributed throughout the continuous liquid medium. As used herein, an aqueous dispersion is an aqueous composition that is a dispersion in which the continuous liquid medium contains 50% or more water by weight based on the weight of the continuous liquid medium. Substances that are dissolved in the continuous liquid medium are considered herein to be part of the continuous liquid medium. The collection of all the dispersed particles is known herein as the “solid phase” of the dispersion. As used herein, an “emulsion” is a dispersion in which the dispersed particles are liquid.

As used herein, the “solids content” of an aqueous composition is the amount of material that remains when water and compounds having a boiling point of 250° C. or less have been removed. Solids content is characterized either by weight percent based on the total weight of the aqueous composition or by volume fraction based on the total volume of the aqueous composition.

Ethylcellulose polymer, as used herein, means a derivative of cellulose in which some of the hydroxyl groups on the repeating glucose units are converted into ethyl ether groups. The number of ethyl ether groups can vary. The USP monograph requirement for ethyl ether content is from 44 to 51%.

As used herein, the viscosity of an ethylcellulose polymer is the viscosity of a 5 weight percent solution of that ethylcellulose polymer in a solvent, based on the weight of the solution. The solvent is a mixture of 80% toluene and 20% ethanol by weight. The viscosity of the solution is measured at 25° C. in an Ubbelohde viscometer.

As used herein, a fatty acid is a compound having a carboxyl group and a fatty group. A fatty group is a linear or branched chain of carbon atoms connected to each other that contains 8 or more carbon atoms. A hydrocarbon fatty group contains only carbon and hydrogen atoms.

When it is said herein that a composition contains “little or no” amount of an ingredient, it is meant that the amount of that ingredient is either zero or is 0.1% or less by weight based on the dry weight of ethylcellulose polymer. When it is said herein that a composition contains “little or no” amount of a list of ingredients, it is meant that the sum of the amounts of those ingredients is either zero or is 0.1% or less by weight based on the dry weight of ethylcellulose polymer. These definitions of “little or no” apply to any composition that contains ethylcellulose polymer, whether the composition is an aqueous composition or is not an aqueous composition.

As used herein, a plasticizer is a compound that is miscible with ethylcellulose polymer, and that, when mixed with ethylcellulose polymer, reduces the glass transition temperature of that ethylcellulose polymer.

A compound is considered herein to be water soluble if 2 grams or more of the compound will dissolve in 100 grams of water at 25° C. A compound is considered water soluble even if it is required to heat the water to a temperature higher than 25° C. in order to form the solution, as long as the solution of 2 grams or more of the compound in water is a stable solution at 25° C.

A “polymer,” as used herein is a relatively large molecule made up of the reaction products of smaller chemical repeat units. Polymers may have a single type of repeat unit (“homopolymers”) or they may have more than one type of repeat unit (“copolymers”). Copolymers may have the various types of repeat units arranged randomly, in sequence, in blocks, in other arrangements, or in any mixture or combination thereof. Polymers have weight-average molecular weight of 2,000 daltons or higher.

Compounds that can react with each other to form polymer are known herein as monomers. Vinyl monomers have one or more carbon-carbon double bond that is capable of reacting with other carbon-carbon double bonds to form polymer. Olefin monomers are hydrocarbon compounds with exactly one carbon-carbon double bond.

The Acid Value (AV) of a compound is measured as follows. The sample is dissolved in a 50/50 blend of xylenes/isopropanol on a hot plate. Once dissolved the solution is then titrated using alcoholic potassium hydroxide solution (concentration 0.1N) to the phenolphthalein endpoint. AV is then calculated by (mL titrant*56.1*N)/g of sample and is reported in units of mg KOH per gram of compound.

As used herein, a dispersant is a compound that improves the ability of the dispersed particles in a dispersion to become dispersed (that is, distributed throughout the continuous liquid medium) and/or to remain dispersed upon exposure to storage at 25° C., exposure to temperatures higher than 25° C., exposure to shear, or a combination thereof. A dispersant that is also a polymer is known herein as a polymeric dispersant. A dispersant that has weight-average molecular weight below 2,000 daltons is known herein as a monomeric dispersant.

As used herein, a base compound is a compound that has the ability to accept a proton to form the conjugate acid of that compound, and the conjugate acid of that compound has pKa of 7.5 or greater.

As used herein, a fugitive base is a basic compound. When an aqueous composition that contains a fugitive base is dried, when the remaining dried composition has 5% or less water by weight based on the weight of the dried composition, the amount of the fugitive base that remains in the dried aqueous composition is 5% or less by weight based on the weight of fugitive base present in the aqueous composition prior to the drying process. A non-fugitive base does not depart from an aqueous composition when the aqueous composition is subjected to a drying process. That is, when an aqueous composition that contains a non-fugitive base is dried, when the remaining dried composition has 5% or less water by weight based on the weight of the dried composition, the amount of the non-fugitive base that remains in the dried aqueous composition is 80% or more by weight based on the weight of non-fugitive base present in the aqueous composition prior to the drying process.

A water-soluble polymer is considered herein to be a neutral water-soluble polymer if the polymer, when present in a solution of water, has no appreciable amount of any covalently bound ionic groups in an ionic state at any pH from 3 to 11. As used herein, “no appreciable amount” means 0 to 0.001 milliequivalents of ionic groups per gram of polymer.

When it is stated herein that a ratio is X:1 or larger, it is meant that the ratio is Y:1, where Y is equal to or larger than X. For example, if it is stated that a certain ratio is 0.2:1 or larger, the ratio may be 0.2:1 or 0.5:1 or 100:1, but the ratio is not 0.1:1 or 0.02:1. Similarly, when is stated herein that a ratio is W:1 or smaller, it is meant that the ratio is Z:1, where Z is equal to or smaller than W. For example, if it is stated that a certain ratio is 5:1 or smaller, the ratio may be 5:1 or 4:1 or 0.1:1, but the ratio is not 6:1 or 10:1.

Any ethylcellulose polymer may be used in the present invention. The ethyl ether content of the ethylcellulose polymer is 44% or more; preferably 47% or more; more preferably 48% or more. The ethyl ether content of the ethylcellulose polymer is 51% or less; preferably 50% or less.

The ethylcellulose polymer preferably has viscosity of 2 mPa-s or higher; more preferably 5 mPa-s or higher; more preferably 12 mPa-s or higher; more preferably 16 mPa-s or higher. The ethylcellulose polymer preferably has viscosity of 120 mPa-s or lower; more preferably 100 mPa-s or lower; more preferably 80 mPa-s or lower; more preferably 60 mPa-s or lower; more preferably 40 mPa-s or lower; more preferably 30 mPa-s or lower.

Commercially available forms of ethylcellulose polymer which may be used in the invention include, for example, those available under the name ETHOCEL™, from The Dow Chemical Company. The ethylcellulose polymers used in the inventive examples are commercially available from The Dow Chemical Company as ETHOCEL™ Standard 4, ETHOCEL™ Standard 7, ETHOCEL™ Standard 10, ETHOCEL™ Standard 20, ETHOCEL™ Standard 45, or ETHOCEL™ Standard 100 with ethyl ether content from 48.0 to 49.5%. Other commercially available ethylcellulose polymers useful in embodiments of the invention include certain grades of AQUALON™ ETHYLCELLULOSE, available from Ashland, Inc., and certain grades of ASHACEL™ ethylcellulose polymers, available from Asha Cellulose Pvt.Ltd.

The present invention involves an aqueous dispersion. Preferably, the continuous liquid medium contains water in the amount, by weight based on the weight of the continuous liquid medium, of 60% or more; more preferably 70% or more; more preferably 80% or more; more preferably 90% or more.

Preferably, the dispersed particles in the aqueous dispersion contain ethylcellulose polymer in an amount, by weight based on the total dry weight of the solid phase, of 40% or more; more preferably 50% or more; more preferably 60% or more. Preferably, the dispersed particles in the aqueous dispersion contain ethylcellulose polymer in an amount, by weight based on the total dry weight of the solid phase, of 90% or less; more preferably 80% or less. A dispersed particle is considered herein to contain both material located on the interior of the particle and material located on the surface of the particle, such as, for example, a dispersant.

The composition of the present invention contains one or more polymeric dispersants. The polymeric dispersant has weight-average molecular weight of 5,000 daltons or more. The polymeric dispersant has acid value of 60 or higher; preferably 80 or higher; more preferably 100 or higher; more preferably 120 or higher. The polymeric dispersant has acid value of 190 or lower; preferably 180 or lower; more preferably 170 or lower.

Preferred polymeric dispersants have pendant carboxyl groups. The amount of carboxyl groups may be characterized by the milliequivalents of carboxyl groups per gram of polymeric dispersant (meq/g). Preferably, the polymeric dispersant has an amount of carboxyl groups of 1.5 meq/g or higher; more preferably 2.0 meq/g or higher; more preferably 2.5 meq/g or higher. Preferably, the polymeric dispersant has an amount of carboxyl groups of 5 meq/g or lower; more preferably 4 meq/g or lower; more preferably 3 meq/g or lower.

Preferred polymeric dispersants have no pendant hydroxyl groups. Preferred polymeric dispersants have no pendant amine groups.

Polymeric dispersants with pendant carboxyl groups are said to be in neutral form when 25 mole percent or less of the carboxyl groups are in the protonated state and not in the anionic state. Preferred polymeric dispersants, when they are in neutral form, are not water soluble.

Preferred polymeric dispersants are olefin copolymers, monoester derivatives of cellulose, and mixtures thereof; more preferred are olefin copolymers. Olefin copolymers are copolymers formed from monomers that include one or more olefin monomer and one or more vinyl monomer that has a pendant carboxyl group. Preferred olefin monomers are ethylene, propylene, butylene, and mixtures thereof; more preferred is ethylene. Preferred vinyl monomers having pendant carboxyl group are acrylic acid, methacrylic acid, maleic anhydride, itaconic acid, and mixtures thereof; more preferred is acrylic acid. Also included in the category of olefin copolymers are copolymers formed from monomers that include one or more olefin monomer and one or more vinyl monomer that has a reactive group, where, after polymerization, the reactive group is converted by a chemical reaction to a carboxyl group. Carboxyl groups may be in protonated form, anion form, or salt form.

Monoester derivatives of cellulose are derivatives of cellulose in which one or more six-carbon cycle in the cellulose polymer chain has a pendant group that terminates in structure I:

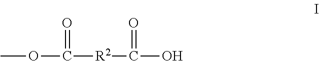

where structure I is attached to a carbon atom that is either a member of a six-carbon cycle in the cellulose polymer chain or else is a member of a group pendant on such a six-carbon cycle. A preferred monoester derivative of cellulose is hydroxypropylmethylcellulose acetate succinate.

Preferably, the weight ratio of ethylcellulose polymer to polymeric dispersant is 1.5:1 or higher; more preferably 1.8:1 or higher; more preferably 2:1 or higher. Preferably, the weight ratio of ethylcellulose polymer to polymeric dispersant is 8:1 or lower; more preferably 4:1 or lower; more preferably 3:1 or lower.

Preferably, the composition of the present invention contains little or no oleic acid. That is, preferably the sum of the weights of all oleic acid, oleate ion, and oleate salts is 0 to 0.1% based on the weight of ethylcellulose polymer. More preferably, the sum of the weights of all non-polymeric fatty acids, non-polymeric fatty acid carboxylate ions, and non-polymeric fatty acid carboxylate salts is 0 to 0.1% based on the weight of ethylcellulose polymer. Preferably, the composition of the present invention contains little or no monomeric dispersant.

In some compositions other than the present invention that contain ethylcellulose polymer, relatively large amounts of a neutral water-soluble polymer are used. Commonly used neutral water-soluble polymers include, for example, polyvinyl alcohol (PVA), poly(N-vinyl pyrrolidone), and neutral water-soluble polymers that are derivatives of cellulose. In contrast to those other compositions, the composition of the present invention preferably contains little or no PVA. More preferably, the composition of the present invention contains little or no PVA or poly(N-vinyl pyrrolidone). More preferably, the composition of the present invention contains little or no PVA, poly(N-vinyl pyrrolidone), or neutral water soluble polymers that are derivatives of cellulose. More preferably, the composition of the present invention contains little or no neutral water-soluble polymer.

In some aqueous compositions other than the present invention that contain ethylcellulose polymer, relatively large amounts of a plasticizer are present. Typical plasticizers are organic esters having molecular weight of 200 or greater and polyethylene glycols having molecular weight of 200 or greater. Commonly used plasticizers include, for example, triethyl citrate (TEC), dibutyl sebacate (DBS), diethyl phthalate, dibutyl phthalate, polyethylene glycol of molecular weight of 200 or higher, and triglycerides of molecular weight of 200 or higher.

In contrast to those other compositions, the aqueous composition of the present invention preferably either contains no plasticizer or else contains an amount of plasticizer, by weight based on the dry weight of ethylcellulose polymer, of 7% or less; more preferably 3% or less; more preferably 1% or less; more preferably 0.3% or less; more preferably 0.1% or less. Preferably, the composition of the present invention contains little or no triethyl citrate (TEC) or dibutyl sebacate (DBS). More preferably, the composition of the present invention contains little or no TEC, DBS, diethyl phthalate, dibutyl phthalate, polyethylene glycol of molecular weight of 200 or higher, or triglyceride of molecular weight of 200 or higher. More preferably, the composition of the present invention contains little or no polyethylene glycol having molecular weight of 200 or greater or organic ester having molecular weight of 200 or greater. More preferably, the composition of the present invention contains little or no plasticizer.

The preferred process for making the aqueous composition of the present invention is as follows. The process comprises feeding ethylcellulose polymer and a polymeric dispersant into a melt-and-mix zone of an extruder wherein the ethylcellulose polymer and polymeric dispersant are heated and mixed together to form a melt; conveying the melt to an emulsification zone of the extruder in which the temperature and pressure are controlled; feeding a base and water into the emulsification zone wherein the melt is dispersed to form an emulsion; conveying the emulsion to a dilution and cooling zone of the extruder; and feeding water into the dilution-and-cooling zone to dilute and cool the emulsion thereby forming an aqueous dispersion. The general process conditions and equipment which may be used to perform the process are disclosed in U.S. Pat. Nos. 5,539,021 and 5,756,659.

Preferably the emulsion, prior to dilution, has volume fraction of higher than 0.6; more preferably 0.65 or higher. Preferably the emulsion, prior to dilution, has volume fraction of less than 0.74.

The preferred extruder has several zones, including a mixing-and-conveying zone, an emulsification zone, and a dilution-and-cooling zone. Preferably, steam pressure is maintained at the feed end, which is contained in the mixing-and-conveying zone. Preferably, the steam pressure at the feed end is controlled by placing kneading blocks and blister elements before the emulsification zone to create a melt seal. Preferably, steam pressure is maintained at the outlet, which is contained in the dilution-and-cooling zone. Preferably, the steam pressure at the outlet is controlled by using a back-pressure regulator. The ethylcellulose polymer is fed into the feed throat of the extruder and flows into the mixing-and-conveying zone. The dispersant(s) (including one or more polymeric dispersants) are also preferably fed into the mixing-and-conveying zone and may be fed separately or jointly. If the dispersant is a solid, it may be optionally fed into the extruder through the extruder feed throat. The polymer phase, which includes the ethylcellulose polymer and the polymeric dispersant, is preferably melted in the mixing-and-conveying zone and conveyed down the barrel of the extruder to the emulsification zone.

In the emulsification zone, the polymer phase is preferably combined with an initial amount of water and a base, to create an emulsion. The emulsion is then preferably conveyed down the extruder and combined with more water in the dilution and cooling zone to form an aqueous dispersion having less than or equal to 60% wt. solids. Preferably, the solids in the aqueous dispersion contain mostly the dispersed particles. That is, the amount of the solids in the aqueous dispersion that is in the form of dispersed particles is preferably 80% or more; more preferably 90% or more; more preferably 95% or more.

Preferably, the base that is fed into the emulsification zone is a fugitive base. Preferred fugitive bases have boiling point of 250° C. or lower; more preferably 200° C. or lower. Preferred fugitive bases have boiling point of −80° C. or higher. Preferred fugitive bases have pKa of the conjugate acid of 8 or higher. Preferred fugitive bases have pKa of the conjugate acid of 35 or lower. Preferred bases are ammonia, fugitive amine bases, and mixtures thereof; more preferred are ammonia, morpholine, alcohol amines, and mixtures thereof; more preferred are ammonia, morpholine, diethanolamine, 2-amino-2-methyl-1-propanol, and mixtures thereof; more preferred is ammonia.

Preferably the ratio of the moles per minute of base being fed to the extruder to the moles per minute of carboxyl groups on the polymeric dispersant being fed to the extruder is 0.8:1 or higher; more preferably 0.9:1 or higher. Preferably the ratio of the moles per minute of base being fed to the extruder to the moles per minute of carboxyl groups on the polymeric dispersant being fed to the extruder is 2:1 or lower; more preferably 1.75:1 or lower.

The aqueous composition of the present invention preferably has pH of 12 or lower; more preferably 11 or lower; more preferably 10 or lower. The aqueous composition of the present invention has pH of 8 or higher.

The dispersed particles in the aqueous composition of the present invention preferably have volume-average particle diameter of 3 micrometers or less; more preferably 2 micrometer or less. The dispersed particles in the aqueous composition of the present invention preferably have volume-average particle diameter of 50 nm or greater; more preferably 100 nm or greater. Particle size was measured using laser diffraction. A suitable instrument is a COULTER™ LS-230 or COULTER™ LS-13-320 particle size analyzer (Beckman Coulter Corporation).

The viscosity of the aqueous composition of the present invention is measured at 25° C. using a Brookfield RV-II viscometer with an RV2 or RV3 spindle spinning at 50 rpm. The spindle is chosen to give the torque signal nearest to the center of the viscometer's torque range. Preferably the viscosity of the aqueous composition is 100 mPa-s or lower; more preferably 80 mPa-s or lower; more preferably 60 mPa-s or lower; more preferably 40 mPa-s or lower; more preferably 30 mPa-s or lower. Preferably the viscosity of the aqueous composition is 1 mPa-s or higher.

The aqueous composition of the present invention preferably has a solids content, by weight based on the weight of the aqueous composition, of 5% or more; more preferably 10% or more; more preferably 15% or more; more preferably 20% or more. The aqueous composition of the present invention preferably has a solids content, by weight based on the weight of the aqueous composition, of 55% or less; more preferably 50% or less; more preferably 45% or less; more preferably 40% or less; more preferably 35% or less.

A preferred use for the aqueous composition of the present invention is to produce a film. The aqueous composition of the present invention is optionally mixed with additional ingredients; a layer of the aqueous composition of the present invention is applied to a surface, and the water is removed. The resulting film preferably contains residual water in an amount, by weight based on the weight of the film, of 0 to 5%; more preferably 0 to 2%; more preferably 0 to 1%; more preferably 0 to 0.5%.

The resulting film may be used for any purpose. A preferred purpose is as a pharmaceutical coating or a food coating; more preferred is a pharmaceutical coating; more preferred is a modified-release pharmaceutical coating. A preferred method of making a modified-release pharmaceutical coating is to provide a multiparticulate formulation that contains a drug and apply a coating of the film to envelop or encapsulate each of the multiparticulates. Preferred multiparticulates are made from sugar or microcrystalline cellulose and have a drug applied as a layer to the surface or sprayed onto the surface. Alternatively, multiparticulates may contain a drug located in the interior of the particles, for example if the multiparticulates are made by extrusion followed by spheronization of a mixture of the drug with the material that will be made into the multiparticulates. The coating formed by the film made from the aqueous composition of the present invention preferably forms a complete layer of coating on 50% or more of the particles (by number); more preferably, the coating forms a complete layer of coating on 75% or more of the particles (by number). Preferably, on 90% or more of the particles (by number), the coating covers 75% or more of the area of the surface of each particle.

Suitable multiparticles may be pellets, granules, powders, or other forms.

Also contemplated are embodiments in which the film is used as a modified-release coating on pharmaceutical dosage forms such as tablets or capsules.

When an aqueous composition of the present invention is used for making a film, it is preferred to use a plasticizer.

When a plasticizer is used, the plasticizer may be added to the composition at any point during the process of making the composition. For example, plasticizer may be added to the melt and mix zone of an extruder along with the ethylcellulose polymer and the polymeric dispersant. Preferably, the aqueous composition of the present invention is made with little or no plasticizer, and then plasticizer is post added by simple mixing to the aqueous dispersion. For example, aqueous composition of the present invention is preferably made by the extrusion method described above, including dilution, cooling, and removal from the extruder, without the use of plasticizer, and then plasticizer is preferably added to the aqueous composition of the present invention.

When a plasticizer is used, preferred are one or more plasticizers selected from the group consisting of triglycerides, organic esters having molecular weight of greater than 220, and alkyl carboxylic acids. When a plasticizer is used, the amount of plasticizer preferably is, by weight based on the total dry weight of the solid phase, 10% or more; more preferably 15% or more. When a plasticizer is used, the amount of plasticizer preferably is, by weight based on the total dry weight of the solid phase, 40% or less; more preferably 30% or less.

When a composition forms a coating on a plurality of particles, it is desired that the coating have good film properties, such as relatively high values of Young's modulus, tensile strength, and maximum elongation. It is contemplated that these properties may be tested by making a free film (that is, a film that is not attached to any substrate) and testing the tensile properties of the free film. It is contemplated that films that have acceptable properties as free films will also have acceptable properties when coated onto multiparticulates.

The following are examples of the present invention.

Materials used were as follows:
EC=ETHOCELTM STD 20 ethylcellulose polymer, from The Dow Chemical Company.
PD=PRIMACORTM 5980 copolymer, polymeric dispersant, from The Dow Chemical

Company, a copolymer of ethylene and acrylic acid, with approximately 21% polymerized units of acrylic acid, and acid value of 155 mg KOH/g polymer.
DBS=dibutyl sebacate.

### EXAMPLE 1

### Aqueous Dispersion of EC with PD

Using a Berstorff extruder (ratio of length to diameter of 36, diameter of 25 mm), EC was delivered using a Schenck Accurate volumetric solids feeder equipped with a large tube and large open helix, and PD was delivered by a second Schenck Accurate volumetric solids feeder equipped with a large tube and large helix. EC and PD were delivered to a heated melt and mix zone to produce a melt. The melt was conveyed to an emulsification zone and mixed with water and ammonia to produce an emulsion. The barrels were set to 170° C. The front end heaters (3-way valve, back pressure regulator) remained on during the run and set to 180° C. The initial water feed heater (“IA heater”) was turned on to deliver at ˜160° C. The dilution water heater was set to 120° C. Extruder exit temperature was 165° C.

The weight ratio of EC to PD was 70:30. The mole ratio of ammonia to carboxyl groups on the PD was 1.4:1. The solids content of the emulsion was 70% by weight.

Resulting dispersion: pH was 9.27; solids was 26.95% by weight; viscosity (Brookfield RV2, 50 rpm, 25° C.) was 22 mPa-s; volume-average particle diameter was 0.552 μm.

### COMPARATIVE EXAMPLE A

### EC, DBS, and Oleic Acid

Using the same extruder as Example 1, EC was delivered using a Schenck Accurate volumetric solids feeder equipped with a large tube and large open helix. The DBS and oleic acid were blended at a 16.25/8.75 weight ratio and delivered using an ISCO syringe pump that was plumbed into the melt zone. The melt zone was set to 135° C., the emulsion zone was set to 125° C., and the exit zones were set to 145° C. Water and ammonia were introduced into the emulsion zone. The front end heaters (3-way valve, BPR) remained on during the run and set to 180° C. The IA heater was turned on to 150° C. to ensure that it was delivering at least at 130° C. The dilution heater was set to 120° C. Extruder exit temperature was 140° C.

Weight ratios of EC/DBS/Oleic acid were 74/17/9. The mole ratio of ammonia to oleic acid was 1.4:1. The solids content of the emulsion was 82% by weight.

The properties of the resulting dispersion: pH was 9.02; solids level was 27.02% by weight; viscosity (Brookfield RV2, 50 rpm, 25° C.) was 117 mPa-s; volume-average particle size was 0.203 um.

### EXAMPLE 2

### Composition with DBS

The dispersion of Example 1 was mixed with DBS. The amount of DBS was 27% by weight based on the total weight of solids in the dispersion. The liquid DBS was added to the aqueous dispersion and combined with a impeller spinning at 200 rpm for 2 minutes.

### EXAMPLE 3

### Tensile Testing of Films

Films were cast as follows: Films were cast at thickness of 0.5 mm (20 mil) wet onto a pre-cleaned glass plate using a BYK four-sided draw-down bar. Films were covered and transferred to an oven set to 60° C. to cure for 2 hr. Films were then taken to a controlled humidity room (55% relative humidity, 22° C.) for at least 12 hrs for the moisture content of the films to equilibrate.

Tensile measurements were taken using an Instron™ frame 4201 tensile tester using a 50N static load cell (11 lb) equipped with smooth rubber grips. Prior to analysis, the films were held in a controlled humidity room (22° C., 50% RH) and allowed to equilibrate for a minimum of 12 hrs Immediately prior to analysis, the films were removed from the glass substrates using a straight blade to lift and peel the films away from the surface of the glass plate. The films were punched using a pneumatic press using the ASTM D638 type V (dog bone) die. Each type of film was analyzed using ten samples cut from at least three different films. The thickness was determined by measuring along three points of the center of the film strips using a Mitutoya Digimatic™ Indicator and taking the average thickness. The strips were pulled at 0.508 cm/min (0.2 in/min). The Young's modulus was measured by fitting the points in the linear area of the stress/strain curve. The maximum stress (reported as Tensile Strength) and strain at break (reported as % Elongation) were manually determined by reading the values from the stress/strain curve.

Results were as follows:

| | Young's Modulus | Elongation | Tensile Strength |
|---|---|---|---|
| Sample | (MPa) | (%) | (MPa) |
| Comparative Example | 23.1 | 39.0 | 1.3 |
| A | | | |
| Example 2 | 32.6 | 35.6 | 2.1 |

Example 3 had comparable elongation to Comparative Example A, and Example 3 had improved Young's Modulus and Tensile Strength over Comparative Example A.

### COMPARATIVE EXAMPLE B

### Attempts to use PVOH

A procedure using an extruder as in Example 1 was attempted. The ingredients fed to the extruder were EC, vegetable oil plasticizer (VEG-P), and PVOH (MOWIOL™ 18-88 polyvinyl alcohol, 87% hydrolyzed, from Clariant GmbH). No base was fed to the extruder. Feed rates were as follows:

Feed Rates (grams/minute)

| EC | VEG-P | PVOH | Initial Aqueous | Dilution Water |
|---|---|---|---|---|
| 41.2 | 11.4 | 4.2 | 10.0 | 115 |
| 41.2 | 11.4 | 4.2 | 15.1 | 115 |
| 41.2 | 11.4 | 4.2 | 18.9 | 115 |
| 41.2 | 11.4 | 4.2 | 22.1 | 115 |
| 41.2 | 11.4 | 4.2 | 30.9 | 115 |

In all attempts, no dispersion of EC resulted.

1. An aqueous composition having pH of 8 or higher and comprising
(a) a solid phase comprising dispersed particles that comprise an amount of ethylcellulose polymer,
(b) an amount of one or more polymeric dispersants, wherein said polymeric dispersant has a weight-average molecular weight of 5,000 daltons or higher, and wherein said polymeric dispersant has an acid value of 60 to 190 mg KOH/g of polymer.

2. The aqueous composition of claim 1 wherein said composition comprises monomeric dispersant in an amount of 0 to 0.01% by weight based on the weight of said composition.

3. The aqueous composition of claim 1 wherein said polymeric dispersant comprises one or more olefin copolymers, one or more monoester derivatives of cellulose, or a mixture thereof.

4. A method of making the aqueous composition of claim 1, wherein said method comprises
(i) feeding the ethylcellulose polymer and the polymeric dispersant into a melt and mix zone of an extruder wherein the ethylcellulose polymer and the polymeric dispersant are heated and mixed together to form a melt;
(ii) conveying the melt to an emulsification zone of the extruder in which the temperature and pressure are controlled;
(iii) feeding a base and water into the emulsification zone wherein the melt is dispersed to form an emulsion having 60% to 72% solids by volume;
(iv) conveying the emulsion to a dilution and cooling zone of the extruder; and feeding water into the dilution and cooling zone to dilute the high internal phase emulsion thereby forming the aqueous composition.

5. The method of claim 3, wherein said base is a fugitive base.

6. A film made by a process comprising removing water from the aqueous composition of claim 1.

7. The film of claim 5 wherein said film is on the surface of or encapsulating or enveloping a multiparticulate.

8. The film of claim 5 wherein said film is on the surface of a pharmaceutical tablet or pharmaceutical capsule.
